(19) United States
(12) Patent Application Publication
(10) Pub. No.: US 2018/0235603 A1
(43) Pub. Date: Aug. 23, 2018

(54) ACCESS AND SUTURE APPARATUS, SYSTEM, AND METHOD OF CLOSING TISSUE

(71) Applicant: SUTURE EASE, INC., San Jose, CA (US)

(72) Inventor: SCOTT HENEVELD, Whitmore, CA (US)

(21) Appl. No.: 15/899,654

(22) Filed: Feb. 20, 2018

Related U.S. Application Data
(60) Provisional application No. 62/460,162, filed on Feb. 17, 2017.

(51) Int. Cl. A61B 17/04; A61B 17/06; A61B 1/012

(52) U.S. Cl. CPC A61B 17/0469; A61B 17/06; A61B 2017/00637; A61B 1/012; A61B 17/0482

(57) ABSTRACT

A surgical instrument set includes capabilities to obtain access and close the fascial/peritoneal layer at the trocar wound site in a quick, consistent and reproducible manner at the end of a procedure. To facilitate closure of the wound, the system includes a scope guide, an obturator/adaptor, a suture passing needle used in combination with a closure guide apparatus which has suture capture features disposed at or near the distal tip.

### CROSS-REFERENCE TO RELATED APPLICATIONS

This application claims the benefit of priority of U.S. provisional patent application No. 62/460,162, filed Feb. 17, 2017, the contents of which are herein incorporated by reference.

### BACKGROUND OF THE INVENTION

### 1. Field of the Invention

The invention relates generally to surgical instruments. More particularly, the invention relates to surgical instruments for approximation, ligation and fixation of tissue using a suture, and particularly to the approximation of tissue separated by mean of an endosurgical trocar being inserted into a body cavity.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Numerous methods currently exist for performing laparoscopic procedures. One of the more commonly used methods is known as closed laparoscopy which utilizes a sharp needle (e.g., Veress needle) to puncture the abdominal wall and insufflate the abdominal cavity with an inert gas such as carbon dioxide through the needle. This process of insufflating the cavity separates the abdominal wall from the underlying organs creating a gap for the surgeon to work within. A trocar/cannula system is then used to maintain the insufflated cavity and provide a working portal for which instruments can be passed into and out of the abdominal cavity to perform various surgical procedures. When the procedure is completed, it is desirable for the surgeon to close the incision site using suture material to minimize the risk of adverse post-operative events.

One of the post-operative complications associated with this procedure is the incidence of trocar site hernias, where a portion of an organ or fatty tissue protrudes out through the hole in the abdominal wall created by the trocar access portal. It is believed that improper closure, or complete lack of closure, of the incision site at the peritoneum is the primary cause of these hernias which form during the post-operative period ranging from several days to several months following the procedure. Traditional methods of wound site close require an additional set of instruments (suture passers, guides, and the like) to be introduced into the surgery. A number of these instruments have been previously disclosed. However, the prior art related to trocar wound site closure instrumentation are typically cumbersome to use and do not provide for a simple, reproducible, and reliable means of closing the wound site. In addition, when a large tissue specimen is removed from the abdominal cavity, the initial incision site may be enlarged to assist removal of the specimen. Larger incision sites are also encountered when Single Site Laparoscopic Surgery (SSLS) is performed. SSLS procedures are performed where all the surgical instrumentation and the laparoscope are introduced to the abdominal cavity through only one access site. Traditional devices are often not capable of being utilized to close the wound site with the enlarged incision, necessitating a time consuming manual closure of the site.

Previously disclosed was a dual functioning instrument set consisting of a guide and needle that has the capabilities to insufflate the abdominal cavity as well as passing and retrieving suture for closure of the incision site. The needle contains a deployable snare that is used to pass and retrieve the suture. The guide is used to repeatedly locate the needle relative to the inner abdominal wall allowing for consistent placement of sutures. This instrument set may require the aid of an endoscope to view the suture as it is retrieved by the needle in the snare loop.

In view of the foregoing, there is a need for an improved needle and guide instrument set that allows for the surgical procedure to be completed with a device that combines initial access along with subsequent closure for accommodating larger incision sites. This may be advantageous in eliminating a manual closure, thus reducing surgical time and other complications associated with manual closure.

### SUMMARY OF THE INVENTION

Embodiments of the present invention provide an optical obturator/adaptor for laparoscopic procedures comprising a tip configured to pass through tissue; a hollow interior receiving one of a scope guide and a closure guide therein; and a scope rail to facilitate guiding a laparoscope.

In some embodiments, the optical obturator/adaptor has the scope rail positioned along an outer edge of a lateral side wall thereof. While in some embodiments, the optical obturator/adaptor has the scope rail formed as a lumen through the obturator/adaptor.

In some embodiments, the tip is a bladeless tip.

In some embodiments, the optical obturator/adaptor further includes a protrusion disposed on an outer surface of the obturator/adaptor located proximal to a relief where a suture passing needle exits when placing a suture.

In some embodiments, the optical obturator/adaptor further includes a gas port near a distal end of the hollow interior.

In some embodiments, the optical obturator/adaptor is formed from a translucent or transparent material for optically viewing through a distal end thereof.

Embodiments of the present invention further provide a system for laparoscopic surgery comprising an obturator/adaptor comprising a tip configured to pass through tissue, a hollow interior, and a scope rail to facilitate guiding a laparoscope; and a scope guide receivable at least partially into the hollow interior of the obturator/adaptor, the scope guide comprising a central lumen for passage of a scope, and a fitting communicating with an inflation lumen.

In some embodiments, the central lumen and the inflation lumen are a single lumen.

In some embodiments, the scope guide further comprises an inflation seal preventing gas flow from exiting a top end of the central lumen when the scope is inserted therethrough.

In some embodiments, the scope guide further comprises a lower seal pressing against a surface of the obturator/adaptor when the scope guide is securely positioned in the obturator/adaptor.

Embodiments of the present invention also provide a method for performing laparoscopic surgery comprising inserting an obturator/adaptor through tissue for access to an abdominal cavity, the obturator/adaptor comprising a tip configured to pass through tissue, a hollow interior, and a scope rail to facilitate guiding a laparoscope; mounting a scope guide into the obturator/adaptor, the scope guide comprising a central lumen for passage of a scope, and a fitting communicating with an inflation lumen; connecting the fitting a gas insufflation source and, when the distal end of the obturator/adaptor is positioned into the abdominal cavity where a gas port exit is beyond the peritoneum, insufflating the abdominal cavity; removing the scope guide while the obturator/adaptor remains in the tissue; mounting a closure guide in the obturator/adaptor; releasing snare loops from the closure guide; placing a suture into each of the snare loops with a suture passer; retracting the snare loops with suture ends captured therein against the closure guide; and removing the closure guide to release the suture ends outside of the obturator/adaptor.

In some embodiments, the method further includes passing an articulating scope along the scope rail for visualizing the placement of the suture into each of the snare loops.

In some embodiments, the method further includes keeping the suture ends untied while performing the laparoscopic surgery.

In some embodiments, the method further includes tying ends of the suture to close the site.

These and other features, aspects and advantages of the present invention will become better understood with reference to the following drawings, description and claims.

### BRIEF DESCRIPTION OF THE DRAWINGS

Some embodiments of the present invention are illustrated as an example and are not limited by the figures of the accompanying drawings, in which like references may indicate similar elements.

FIG. 1 illustrates the port site closure system including a guide and a suture passing needle according to exemplary embodiments of the present invention;

FIGS. 2A and 2B illustrate the construct of the guide of FIG. 1;

FIGS. 3A and 3B illustrate the taper feature of the guide channel of the guide of FIG. 1;

FIGS. 4A and 4B illustrate the guide of FIG. 1 with the snares retracted;

FIG. 5 illustrates the continuous radius profile of the snare section of the monolithic snare member;

FIG. 6 illustrates the locking leg feature of the actuator section of the monolithic snare member;

FIG. 7 illustrates the elements of the trocar system according to exemplary embodiments of the present invention;

FIG. 8 illustrates the flexible needle seated in the cannel of the obturating guide;

FIG. 9 illustrates the obturating guide seated in the trocar cannula;

FIG. 10 illustrates the tip of the obturating guide with a typical non-bladed trocar design;

FIGS. 11A and 11B illustrate the flexible needle extending from the guide channel with a clearance slot in the trocar cannula;

FIG. 12 illustrates the trocar system in the abdominal wall with the suture passing needle and suture placed through the tissue;

FIGS. 13A and 13B illustrate relief in the obturating guide to enable the edge of the cannula to catch against the fascia;

FIGS. 14A and 14B illustrate the elements of the trocar system;

FIGS. 15A through 15C illustrate the distal end of the obturating guide;

FIG. 16 illustrates the trocar system in the abdominal wall with the distal end of the placed into the abdominal cavity;

FIGS. 17A through 17C illustrate the distal end of the trocar system in the abdominal wall at different stages;

FIGS. 18A and 18B illustrate the trocar system retrieving the suture;

FIG. 19 is an image of a commercially available wound retractor used in laparoscopic surgery;

FIG. 20 is an image of a commercially available single site multi-port system for laparoscopic surgery;

FIG. 21 is an image of a commercially available articulating laparoscope;

FIG. 22 is an image of the scope guide;

FIG. 23 is an image of obturator/adaptor;

FIG. 24 illustrates the scope guide securely mounted in the obturator/adaptor and being penetrated through the tissue for access; and

FIG. 25 illustrates the closure guide securely mounted in the obturator/adaptor and the suture passer placing suture.

Unless otherwise indicated illustrations in the figures are not necessarily drawn to scale.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiments illustrated by the figures or description below.

As is well known to those skilled in the art, many careful considerations and compromises typically must be made when designing for the optimal configuration of a commercial implementation of any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Broadly, embodiments of the present invention provide a surgical instrument set that may have the capabilities to obtain access and close the fascial/peritoneal layer at the trocar wound site in a quick, consistent and reproducible manner at the end of the procedure. To facilitate closure of the wound, the system includes a scope guide, an obturator/adaptor, a suture passing needle used in combination with a closure guide apparatus which has suture capture features disposed at or near the distal tip.

The needle apparatus may be similar to the apparatus described in prior U.S. Pat. No. 9,393,011, the contents of which are herein incorporated by reference, and as described below.

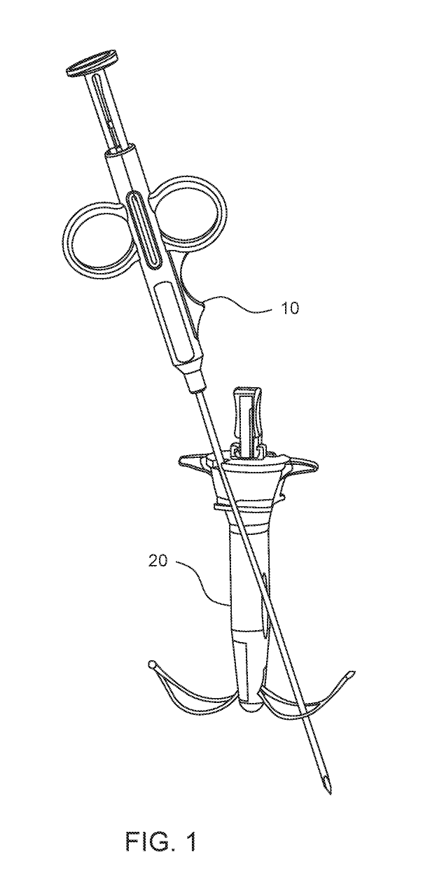

The needle apparatus may be able to function like a suture passer 10 (see FIG. 1), in that it has the ability to carry and retrieve suture through tissue layers for suturing closed the wound site. The needle apparatus may comprise several components including a handle, actuation mechanism, a capture rod, an outer needle shaft, and a spring-loaded safety tip on a hollow obturator tube.

In an exemplary embodiment, a handle at the proximal end of the needle apparatus allows for single-handed or double-handed use. The handle may also contain a finger loop or loops for additional security while holding the needle. An actuator mechanism may be disposed adjacent to the handle and configured for the deployment and retraction of the capture rod used to secure the suture material within the tip of the needle. The actuator mechanism may include a sliding plunger that translates along the long axis of the handle that moves the capture rod between a first position in an extended configuration and a second position in a retracted configuration. The actuator may be spring loaded in one direction such that the capture rod is biased in the retracted position. This may allow the suture to be passively captured without actuation of the plunger. The handle and actuator may be constructed from metals (such as stainless steel, titanium or aluminum) or plastics (such as polyacetal, nylon, polypropylene, poly-ether-ether-ketone, or polycarbonate), or any combination of the two.

A long outer needle shaft may be connected to the proximal handle and extends distally over a length that may range from 2-38 centimeters, or more specifically 10-20 centimeters. The outer needle shaft may have a sharp tip, or needle peak, at the distal-most point to ease the insertion of the needle through the various tissue layers. The outer shaft may house an obturator tube that has a hollow, unobstructed inner lumen, with a blunt tip. The obturator tube may also house a capture rod used for securing the suture for passing through tissue. The outer needle shaft, obturator tube and capture rod would optimally be constructed from metals such as stainless steel, titanium or aluminum.

The distal-most end of the obturator tube may have a blunt or rounded plug or surface at the tip. The entire obturator tube may be spring loaded to allow for the blunt tip to translate away from the tip of the needle when it is loaded, and passively travel back to the tip of the needle when it is unloaded. The obturator spring may be housed within the handle. The spring-loaded obturator would serve as a safety mechanism for protecting the internal organs within the abdomen after the needle is passed through the abdominal wall.

A portion of the wall of the outer needle shaft may be cutout near the distal tip which may be used to create a slot to accommodate the suture during the suture passing process. Similarly, a portion of the wall of the obturator tube may be cut out near the distal tip of the tube to provide an opening for the capture rod to secure the suture to the wall of the outer needle shaft. The window cutout in the obturator tube must be long enough such that it can accommodate the suture as it travels back and forth. Lastly, the capture rod has a slot with one or more ramped faces. A distal ramped surface on the capture rod slot is used to capture the suture against the outer needle shaft. A proximal ramped surface may assist in pushing the suture out of the window in the obturator tube, facilitating the release of the suture from the needle.

The needle capture rod is used to secure the suture to the needle for suture passing activities. Initially the actuator may be pressed to extend the capture rod and expose the slot in the capture rod. A section of suture may be placed into the slot, and the actuator is released to retract the capture rod. As the capture rod retracts, the suture becomes trapped between the distal surface of the slot in the capture rod and the cutout in the outer needle shaft. When the suture needs to be released, the actuator may be pressed again to extend the capture rod. As the capture rod is extended the proximal face of the slot may push the suture material out of the cutout in the obturator tube and away from the needle shaft.

The above describes an exemplary suture passing needle, as described in U.S. Pat. No. 9,393,011, that may be used in embodiments of the present invention. Of course, the present invention is not meant to be limited by any of the specifics described above with respect to the suture passing needle and similar embodiments that perform the same function may be used in various embodiments of the present invention.

The guide apparatus 20, as shown in FIG. 1, may serve dual purposes, as it first may be used to guide the needle 10 through the abdominal wall in a repeatable manner, and second, used to capture the suture material after it is passed into the abdominal wall. The guide 20 is an improvement over the apparatus described in U.S. Pat. No. 9,393,011, as noted below. The guide of U.S. Pat. No. 9,393,011 may comprise a slotted barrel, collapsible barrel tip, plunger, main shaft, cap, suture capturing snare cord, and guide tubes along with various fasteners and springs.

The slotted barrel may have two slotted channels to accommodate the passage of the needle. The two channels may be spaced 180 degrees radially apart from each other such that the stitch can be placed on opposing sides of the wound. The purpose of the channel is to guide the needle repeatably through the same tissue thickness and into the suture snare cord, where the suture can be released. The trajectory of the channels is referenced off the inner wall of the peritoneum such that approximately 5-15 millimeters of tissue bite is achieved from the periphery of the wound. The proximal ends of the channels may have a widened and or tapered opening to ease the entry of the needle into the channels.

As described in U.S. Pat. No. 9,393,011, the guide may comprise a main shaft that is slidably disposed within the slotted barrel of the guide. The main shaft may be used to actuate the expanding arms, comprising living hinges in the preferred embodiment on the collapsible barrel tip. One or more expanding arms may be used to locate the guide against the inner peritoneal wall as a reference point to ensure consistent tissue bite depth of the needle, as previously described. The main shaft may be spring loaded in a proximal position such that the expanding arms are biased to a radially expanded position where the outer profile of the arms exceeds the diameter of the slotted barrel. As the main shaft moves distally, the arms may be contracted such that aligns their outer diameter with the outer diameter of the slotted barrel in a continuous slender fashion. The main shaft may be connected at the distal end to the barrel tip, and connected to the plunger on the proximal end.

The distal end of the barrel tip may have a blunt tip to minimize the potential of harm or damage to the adjacent tissues during insertion. Moving proximally away from the blunt tip, the outer wall of the barrel tip may have a tapered region that gradually radially increases to the outer profile of the guide as designated by the outer diameter of the slotted barrel. The tapered section may facilitate the ease of insertion of the guide into the trocar wound site.

The barrel tip may have one or more stop tabs that provide a hard stop for the barrel tip as the expanding arms are actuated, to prevent excessive flexion in the hinge material. Along the length of the stop tabs, a cutout section may exist for the snare loop to be retracted into for capturing the suture material against the guide.

The guide may have a plunger that is used to actuate the snare cord material. The plunger may be slidably disposed on the slotted barrel. Two snare cords may be connected at their ends to the plunger body, with a loop formed at the distal tip of the guide. The plunger may be spring loaded such that the snare cords are biased into an extended position. As the plunger is pulled proximally, the snare cords are retracted against the extension arms of the barrel tip. As the plunger is released distally, the snare cords are extended out and away from the barrel to create two snare loops for the suture to be passed into. The plunger may have two tabs that can be used to pull the slider proximally using one or more fingers on each tab. The snare cords may be constructed from a mono- or multi-filament wire that has the flexibility to easily bend and conform to various geometries yet stiff enough to create a self-supported snare loop that extends generally perpendicular to the long axis of the guide. Materials that may be used to construct the snare cord include, as exemplary embodiments, plastics such as nylon, polyethylene, polyester or polypropylene or metals such as stainless steel or nitinol.

A plunger at the proximal end of the guide may be used to provide a counterforce when pulling on the slider. As the plunger is pushed and the slider is simultaneously pulled, the snares will move into the retracted position, and the expanding arms are retracted into the slender configuration. As the slider is released, the spring forces will extend the snares to the extended position and the expanding arms will be converted to the radially expanded condition.

In another embodiment, the snares may include a basket element to prevent the needle from traversing deeply into the abdominal cavity and causing potential harm.

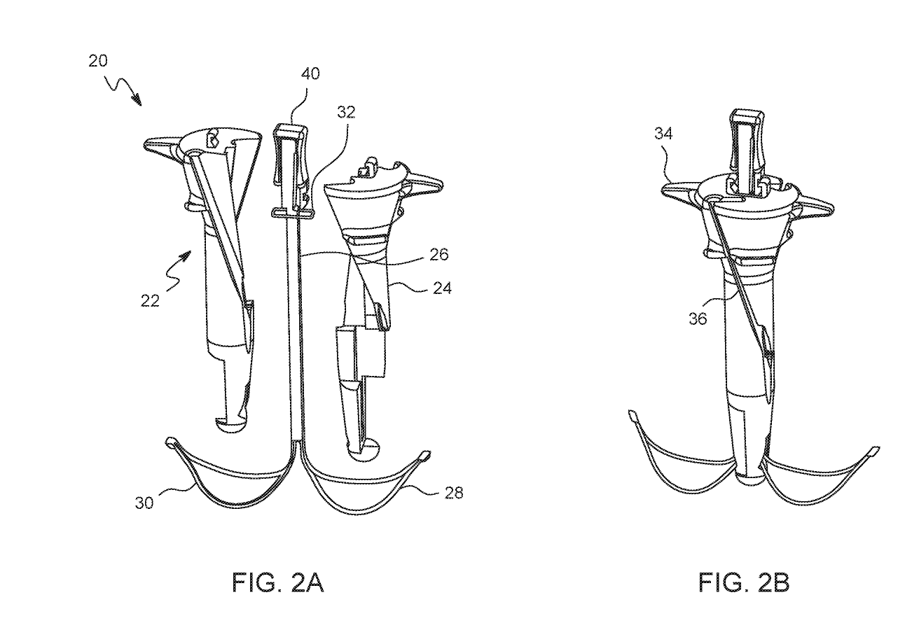

Referring now to FIGS. 1 through 6, in some embodiments, the guide 20 may be formed from a limited number of components, thus providing an economic benefit. In an exemplary embodiment, the guide 20 may be formed of four components as illustrated in FIG. 2A, a monolithic snare member 28, 30, two barrel halves 22, and a clip 32.

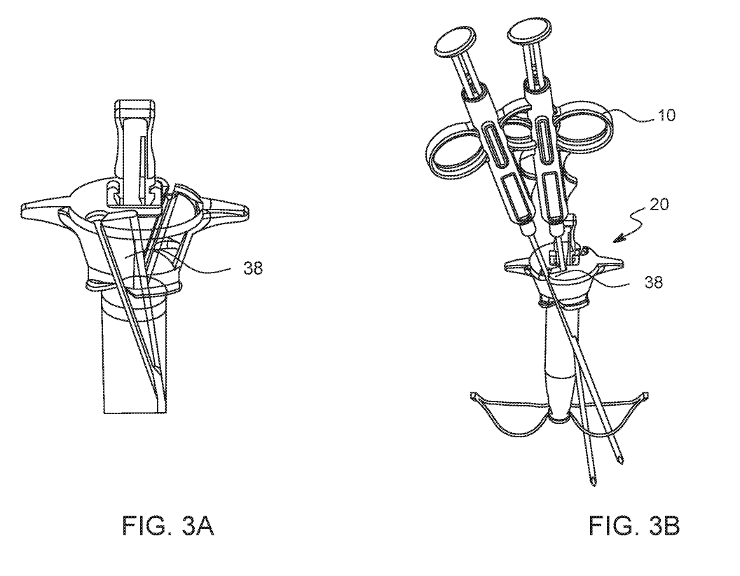

Referring to FIGS. 2A and 2B, the barrel halves 22 can assemble together to provide a construct that may have two slotted channels 36 to accommodate the passage of the needle. The clip 32 may be used to secure the halves together as shown, however, other joining methods are contemplated within the scope of the present invention, such as adhesive bonding, ultrasonic welding, or some other method for joining plastic components. The two slotted channels 36 created by the barrel halves 22 may be spaced 180 degrees radially apart from each other such that the stitch can be placed on opposing sides of the wound, as described below. The purpose of the slotted channels 36 is to guide the needle repeatably through the same tissue thickness and into the suture snare 28, 30, where the suture can be released. The trajectory of the channels 36 is referenced off the inner wall of the peritoneum such that approximately 5-15 millimeters of tissue bite is achieved from the periphery of the wound. The proximal ends 46 of the channels 36 may have a widened and or tapered opening 38 to ease the entry of the needle into the channels 36. Slots 48 in the channels 36 will allow for the middle section of the length of suture to be released from the constraints of the guide channels 36. The width of the slots 48 in the channels 36 should be large enough for the suture to easily be released from the channels 36, yet small enough to not allow the needle to exit the channel or get caught against it. The channels 36 may also provide for added maneuverability of the needle by having a taper feature. As shown in FIG. 3A, the channel 36 can include a taper 38 to create a fulcrum location near the exit of the channel. This allows the user to adjust the needle angle of travel if desired as illustrated in FIG. 3B. The guide 20 may also have lateral tabs 34 to provide counterforce when moving the actuation section.

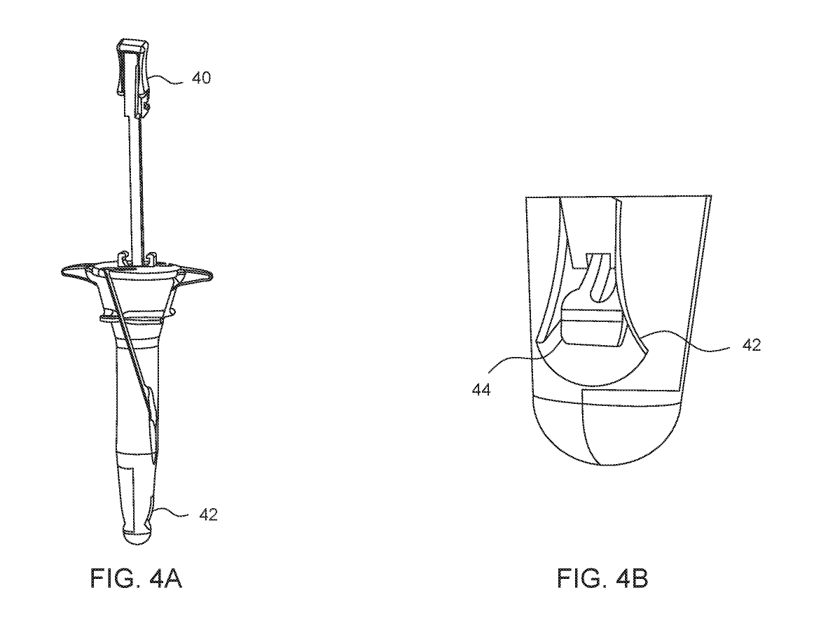

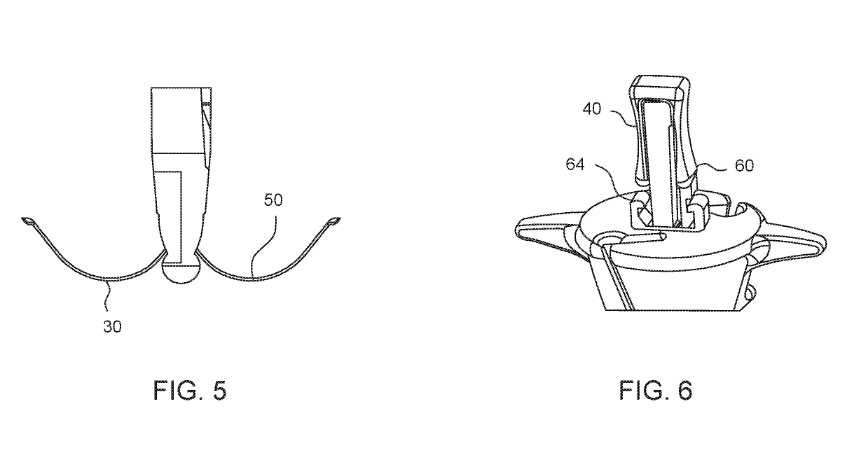

The guide may have a monolithic snare member that includes a shaft section 26 with an actuator section 40 on the proximal end, and two laterally projecting snares 28. 30 on the distal end. The actuator section 40 is used translate movement, via the shaft section 26, to extend and retract the projecting snare sections 28, 30. The shaft section 26 rides along a lumen of the guide 20. The guide 20 has two opposing lateral exit windows 42 near the distal end for the snare sections 28, 30 to pass through and extend laterally. In the extended position, the actuator section 40 is positioned near the barrel, allowing the projecting snares sections 28, 30 to extend to a lateral position as shown in FIG. 3B, for example, essentially perpendicular to the primary axis of the guide 20. The snare section 28, 30 is configured to produce a loop for allowing the passage of suture carried by a needle, as described below. In the retracted position, the actuator section 40 is pulled away from the barrel as shown in FIG. 4A. The projecting snares sections 28, 30 are collapsed as they are withdrawn into the windows 42 of the guide and further into the barrel lumen. The projecting snare loops 28, 3 converge at the distal end to form a rigid tip 44. The rigid tip 44 is sized larger than the barrel window 4s, as shown in FIG. 4B, and thus prevents the snare sections 28, 30 from being pulled fully inside the barrel. The rigid tips 44 also act as a pinching mechanism to trap the suture against the barrel window surface, securing the suture to the guide. The monolithic snare sections 28, 30 may be constructed from a polymer with a high percent of strain to yield property. The snare sections 28, 30 may have thin cross-sections to provide the flexibility to easily bend and conform to various geometric shapes yet stiff enough to create a self-supported snare loop that extends generally perpendicular to the long axis of the guide. The snare sections 28, 30 may also extend laterally in a geometry that from a side view is a continuous radius 50 as shown in FIG. 5. The continuous radius 50 effectively keeps the rigid tip along the “continuous radius” path as the snare section is retracted into the barrel. Materials that may be used to construct the monolithic snare sections 28, 30 include plastics such as nylon, polyethylene, polyester or polypropylene.

The actuation section 40 of the monolithic snare member may have a locking leg 60 that snap fits into a mating feature 64 in the barrel as shown in FIG. 6. The locking leg 60 can be depressed to flex from the “locked” position to enable movement of the monolithic snare sections 28, 30.

The basic procedural steps of the utilization of the suturing system may flow as follows. At the end of the surgical procedure, the trocar is removed from the body exposing the wound. The actuator section 40 of the monolithic snare member is pulled away from the barrel to retract the snare sections 28, 30 such that the profile of the guide is at its minimum. The guide can then be inserted into the wound. The actuator section 40 of the monolithic snare member is then pushed back toward the barrel to deploying the snare sections 28, 30. The guide can be pulled upward and away from the body cavity until the rigid tips of the snare section rest against the inner wall of the peritoneum. A short tail at one end of the suture is secured by the needle. The needle, with suture, is then passed through a first needle channel in the guide and is advanced through the guide, tissue and snare, into the abdominal cavity. The needle then releases the suture into the cavity, and is retracted from the body. A second short tail at the second free end of the suture is then secured by the capture rod in the needle. The needle, with suture, is then passed through the second needle channel in the guide and advanced through the guide, tissue and snare, into the abdominal cavity. The needle then releases the suture into the abdominal cavity and removed from the guide and body. The remaining loop of suture outside the body may then be released from each of the slots in the needle tracks. The actuator section of the monolithic snare member is then again pulled away from the barrel to retract the snares, capturing the free ends of suture, and allowing the guide to be removed from the wound, carrying the suture with it. Once outside the body, the snares may need to be deployed enough to release the free ends of the suture. Lastly, a knot may be tied and pushed down into the wound to close the trocar puncture site.

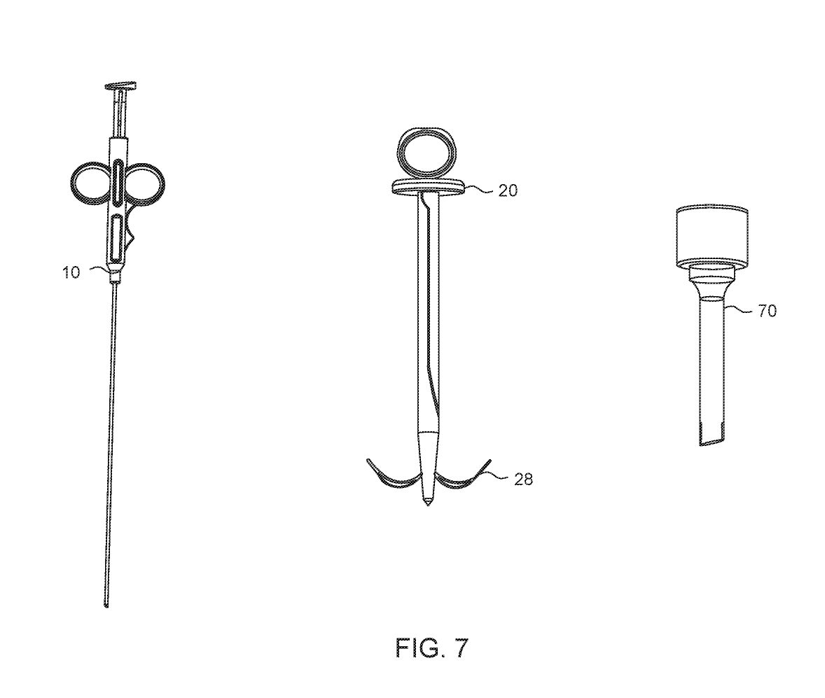

Referring to FIG. 7, in the further improved embodiment, the guide 20 and suture passer 10 are integrated to function with a trocar cannula 70, thus producing a trocar system that enables both laparoscopic access and fascial closure, thus providing economy and ease-of-use to the clinician.

In laparoscopic surgery a trocar device is used the provide access to the abdominal cavity. The trocar is typically comprised to two elements, an obturating trocar, and a cannula through which the obturating trocar fits within. It has long been desired for a trocar device to include a fascial closing functionality to save and time the hassle of removing the trocar cannula and re-entry of the closure device.

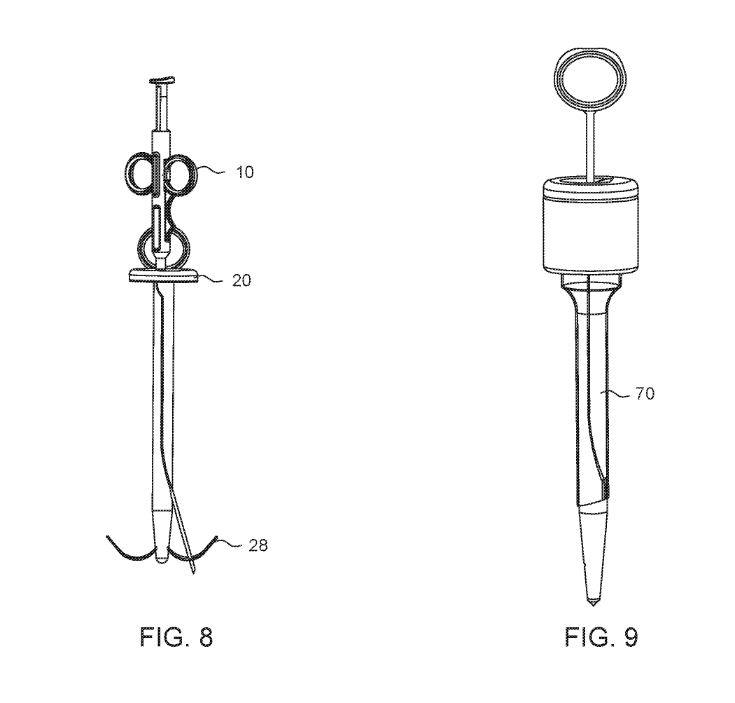

Referring now to FIGS. 7 through 13B, the suture passing needle 10 would have a flexible needle shaft 78 that could traverse the channel 36 within the guide 20. The channel 36 in the obturating trocar/guide 20 could have a trajectory near the exit to direct the needle through the tissue and though the extended snare loop 28 of the guide, as shown in FIG. 8. The radius in the channel path to cause flexing of the needle shaft would be such as to not to interject a strain exceeding about 6% in the needle shaft to prevent permanent deformation of the needle shaft 78. The needle shaft 78 could be constructed of such materials as PEEK, PEI (Ultem), Nylon, or other suitable polymers that have adequate rigidity to penetrate tissue, yet stay below 6% stain when traversing through the channel path. Another suitable material could be super-elastic Nitinol with a strain limit between 3-6%. The needle shaft 78 could also be constructed of stainless steel or other traditional metals (i.e., titanium). Such metal materials could include a “stent” like or struted geometry to keep strain below the 0.2-0.3% range to prevent permanent deformation. The channel 36 in the guide 20 could include the slot 48 to the outer guide surface as identified in FIGS. 2B and 7, for the intent of releasing suture from the guide body. The guide 20 could also include a snare component 28 as identified in FIG. 7 for capturing and retrieval of suture 120.

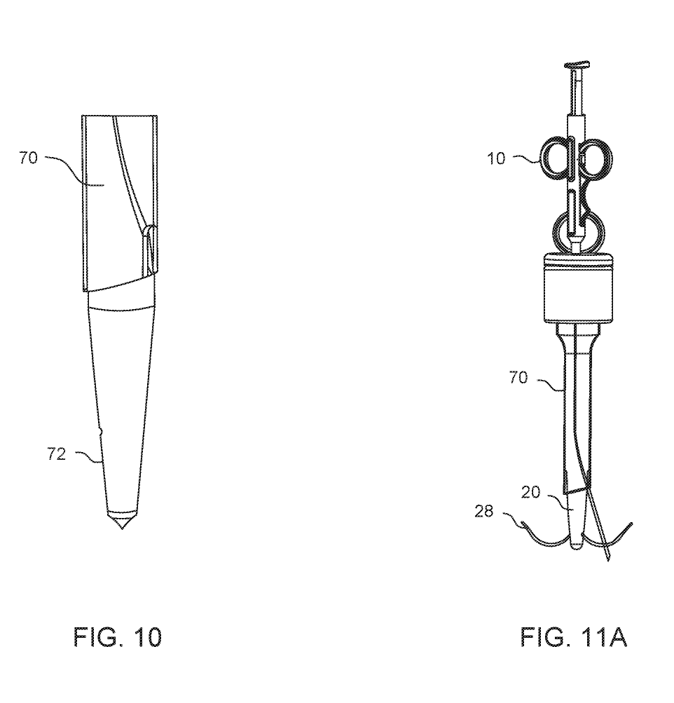

FIG. 9 illustrates the obturating guide 20 seated in the trocar cannula 70. The snares 28 (not visible) are retracted into the guide body to provide a smooth surface for advancing the tip of the guide and trocar cannula 70 through tissue. The tip 72 of the guide 20 could be similar in design to a non-bladed trocar, able to be advanced and transect through tissue in the typical trocar entry fashion, as seen in FIG. 10.

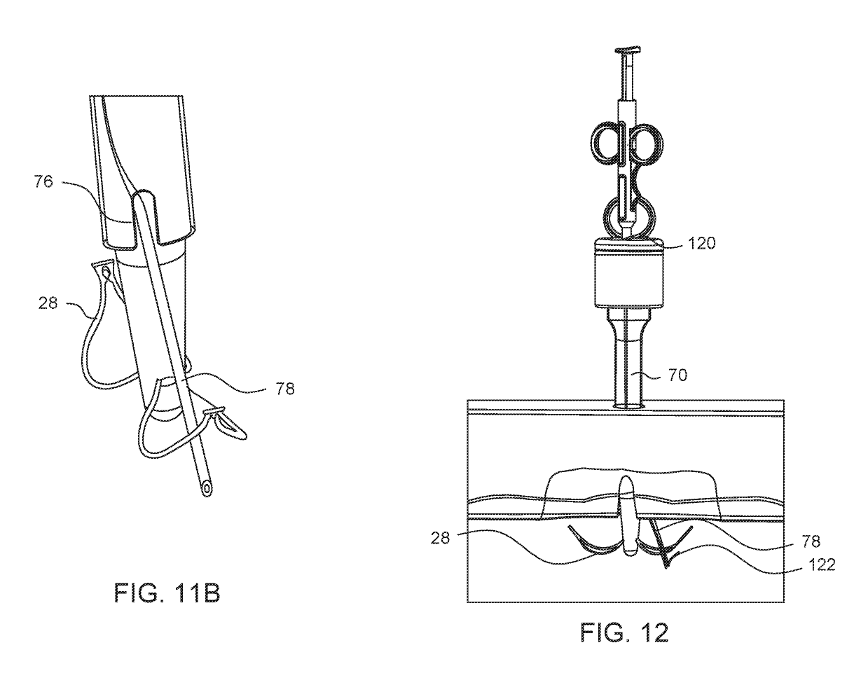

FIGS. 11A and 11B illustrates the trocar device with snares 28 extended, and the suture passing needle 78 fully inserted into the guide channel. The trocar cannula 70 could have contra-lateral slots 76 at the distal end to provide clearance for the suture passing needle 78 to protrude into the tissue and then consequently through the snare loop 28, as shown in FIG. 11B.

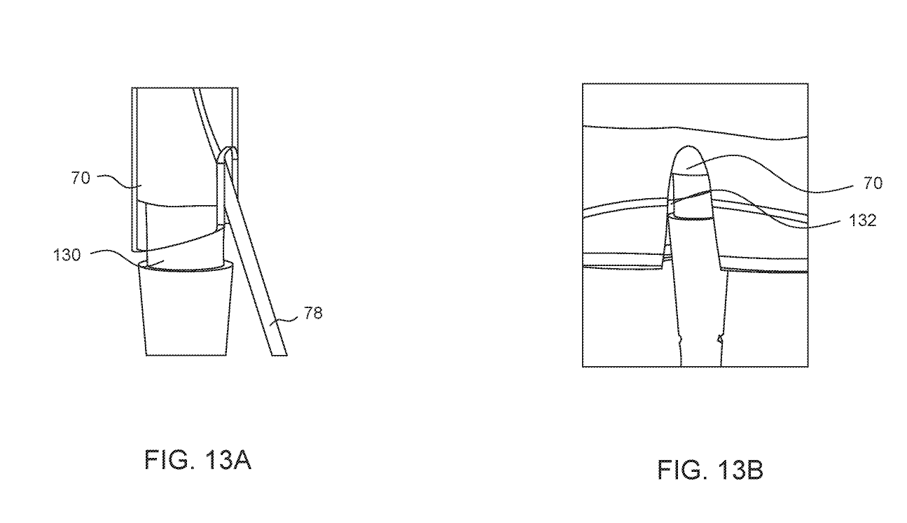

FIG. 12 illustrates the trocar cannula 70 in the abdominal wall, with snares 28 extended, and the suture passing needle 78 carrying suture 120, fully inserted into the guide 20, extending through the tissue, and then through the snares 28. The obturating guide 20 could have a relief 130 near the distal end, as shown in FIG. 13A. The relief 130 would allow for exposure of the edge 132 of the trocar cannula 70 that could be advanced through adipose tissue (fat), but would catch against elastic tissue (fascia and muscle), as shown in FIG. 13B. This would enable the device to be positively positioned relative to the fascia. When desired for passing the trocar cannula 70 through the elastic tissue, the relief 130 of the obturating guide 20 could be covered by slightly retracting the obturating guide relative to the trocar cannula 70, thus resulting in a smooth transition from the trocar cannula 70 to the guide 20.

One sequence of using the above described embodiment could be as follows:
1) Retract snares 28 inside obturating guide 20;
2) Position obturating guide 20 inside trocar cannula 70;
3) Make incision in skin at site;
4) Place trocar device (including guide 20 and trocar cannula 70) through tissue in traditional manner;
5) Remove obturating guide 20 from trocar cannula 70;
6) Utilize trocar cannula 70 per standard laparoscopic technique;
7) Re-enter obturating guide 20 into trocar cannula 70;
8) Extend snares 28;
9) Load suture 120 in suture passing needle 78. Place 1^{st} end 122 of suture 120 through tissue via guide channel 36. Release suture 120 and retract suture passing needle 78;
10) Load the other end of suture in suture passing needle 78. Place 2^{nd} end of suture through tissue via contra-lateral guide channel 36. Release suture 120 and retract suture passing needle 78;
11) Retract snares 28 to capture suture ends 122;
12) Remove entire device to retrieve suture ends 122; and
13) Tie knot to secure suture.

A second sequence of using the above described embodiment could be as follows:
1) Retract snares 28 inside obturating guide 20;
2) Position obturating guide 20 inside trocar cannula 70;
3) Make incision in skin at site;
4) Place trocar device through tissue in traditional manner;
5) Extend snares 28;
6) Load suture 120 in suture passing needle 78. Place 1^{st} end 122 of suture 120 through tissue via guide channel 36. Release suture 120 and retract suture passing needle 78;
7) Load the other end of suture 120 in suture passing needle 78. Place 2^{nd} end of suture through tissue via contra-lateral guide channel 36. Release suture 120 and retract suture passing needle 78;
8) Retract snares 28 to capture suture ends 122;
9) Remove the obturating guide 20 to retrieve suture ends 122;
10) Secure suture ends 122 with hemostat for closure at end of procedure;
11) At end of procedure, remove trocar cannula 70; and
12) Tie knot to secure suture.

Another sequence of using the above described embodiment could be as follows:
1) Retract snares 28 inside obturating guide 20;
2) Position obturating guide 20 inside trocar cannula 70, with the guide 20 slightly retracted to cover the relief 130 near the distal end of the guide 20;
3) Make incision in skin at site;
4) Place trocar device through tissue in traditional manner;
5) Remove obturating guide 20 from trocar cannula 70;
6) Utilize trocar cannula 70 per standard laparoscopic technique;
7) Re-enter obturating guide 20 into trocar cannula 70, fully seated, thus exposing the relief 130 near the distal end of the guide 20;
8) Extend snares 28;
9) Slightly retract the entire device so the edge 132 of the cannula 70 is in the adipose tissue (fat). Push the entire device into the tissue until the edge 132 of the cannula 70 catches against the elastic tissue;
10) Load suture 120 in suture passing needle 78. Place 1^{st} end 122 of suture 120 through tissue via guide channel 36. Release suture 120 and retract suture passing needle 78;
11) Load the other end of suture 120 in suture passing needle 78. Place 2^{nd} end of suture through tissue via contra-lateral guide channel 36. Release suture 120 and retract suture passing needle 78;
12) Retract snares 28 to capture suture ends 122;
13) Remove entire device to retrieve suture ends 122; and
14) Tie knot to secure suture.

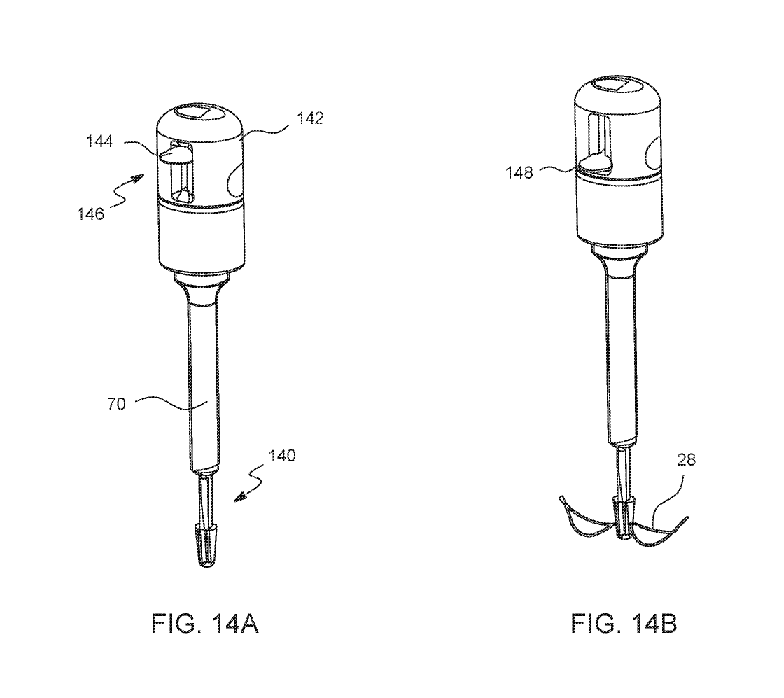

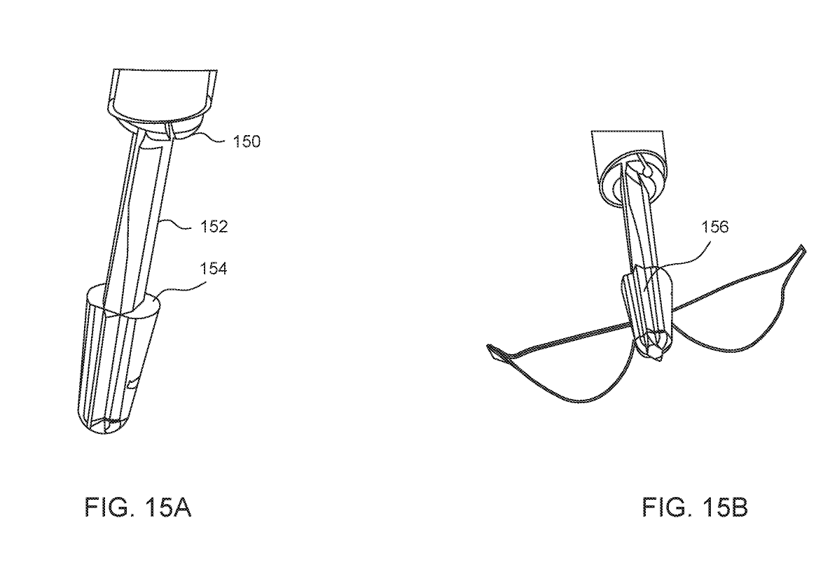

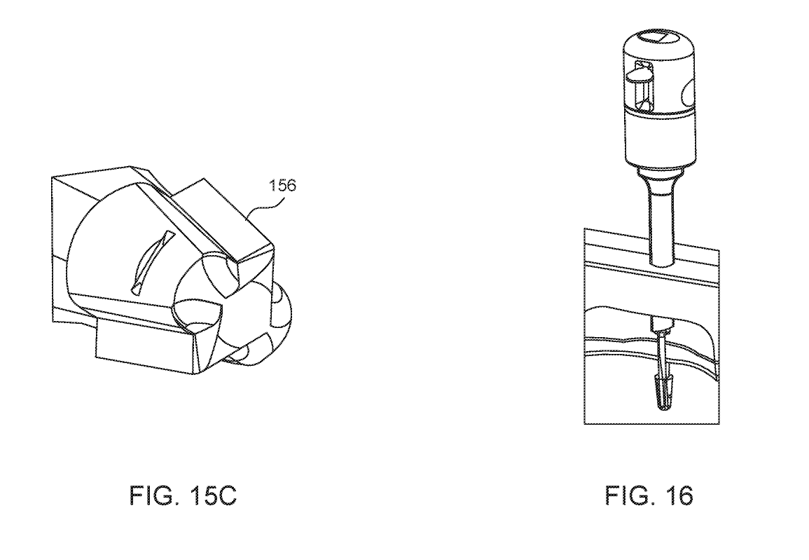

In another embodiment similar to that described above, an obturating guide 20A has a thinned down section 140 on the distal end as shown in FIGS. 14A and 14B. FIG. 15A reveals the proximal edge of the thinned down section to have a blunt dilating surface 150 to facilitate passage through tissue. The distal edge is a shelf/step 154 to catch against tissue, and the mid-section is a relief 152 to receive tissue. Beyond the thinned down section is a distal tip 158 that could have a fin 156 or semi-sharp edge, as shown in FIGS. 15B and 15C, for creating/dissecting a defect in the fascial that is oriented perpendicular (or at 90 degrees) to the snares 28, and thus effectively the penetration locations of the needle for placement of the suture. The fins 156 could be a movable component that rotates on a pivot to extend laterally to provide more of a shelf/step for catching against tissue. The movable fins could be activated to extend and retract upon the axial movement of the snare shaft. When the snares 28 are retracted, the fins 156 are retracted. When the snares 28 are extended, the fins 156 are extended.

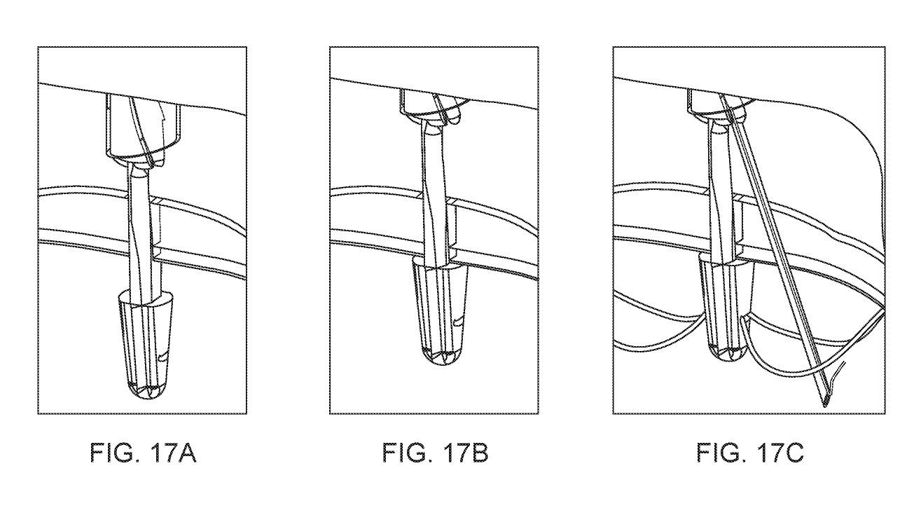

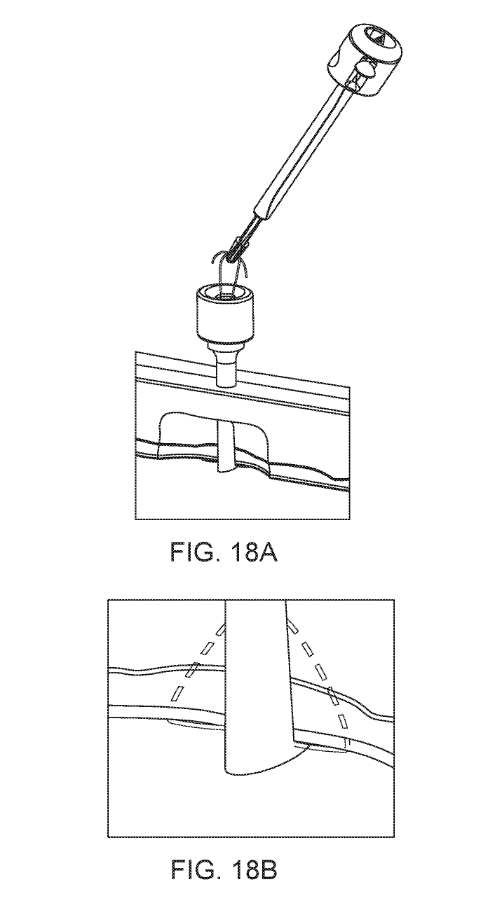

FIG. 16 illustrates the first step of positioning the trocar assembly into the defect. The trocar is entered until the “shelf/step” is inside the abdominal cavity, beyond the peritoneum as shown in FIG. 17A. Step 2 could be pulling upward on the trocar system until the “shelf/step” is seated against the peritoneum as shown in FIG. 17B. The snares could then be expanded. If the fins are movable, the fins and snares could be expanded prior to pulling up on the trocar assembly to seat it against the peritoneum. FIG. 17C shows the suture passer driving the suture through the tissue and then placing it within snare loop. Both sutures tails could be placed as described above, and the snare retracted to capture the suture. The next step could be to push the trocar assembly deeper into the defect until the tip distal edge of the cannula enters the abdominal cavity, beyond the peritoneum, thus enabling the obturating guide to be removed freely without catching the “shelf/step” on the tissue as shown in FIGS. 18A and 18B. The suture would then be released from the guide, and the cannula removed from the defect site to expose the suture, where a knot could be tied to close the wound site.

According to another embodiment of the present invention, a device can include four main components; 1) a closure guide, 2) a suture passer, 3) an obturator/adaptor, and 4) scope guide. The guide and passer can be similar to those described above. The obturator/adaptor is similar, yet has several additional features to enable the adaptor and scope guide to perform access functionality for gaining access to the abdominal cavity similar to direct trocar method commonly practiced as an alternative to Veress needle. The access functionality would be uniquely suited for single site laparoscopic surgery, but not limited to single site.

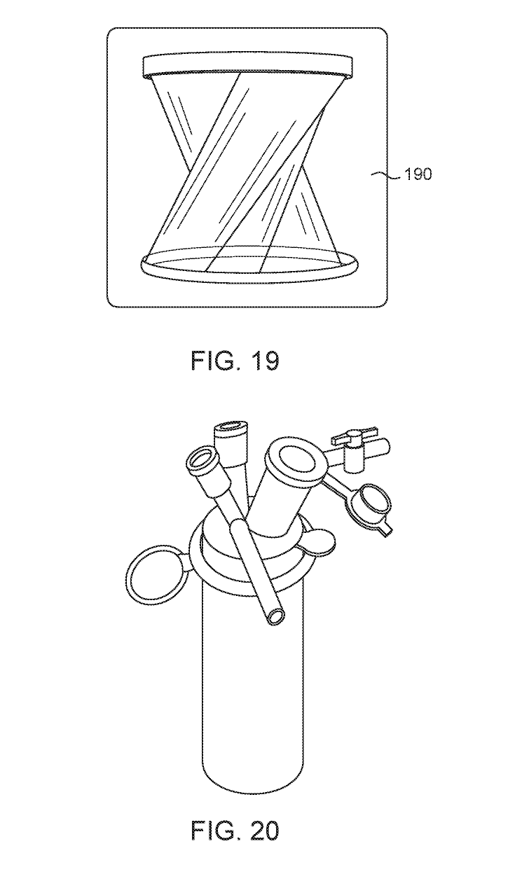

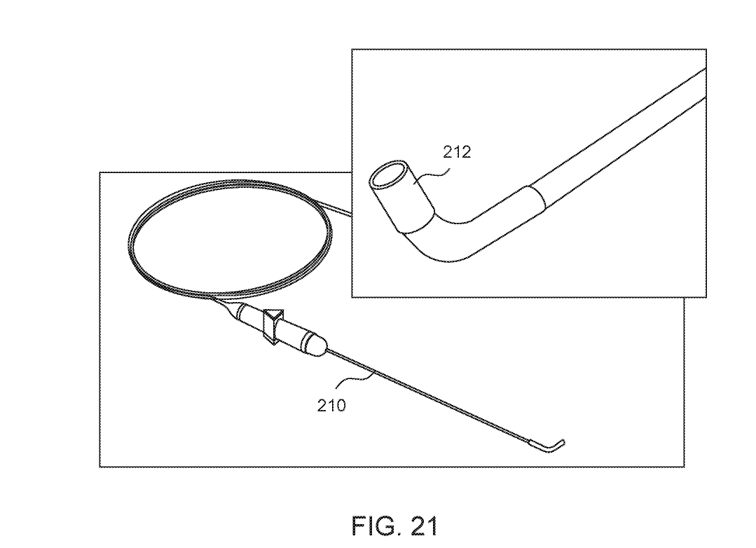

FIG. 19 is an image of a commercially available wound retractor 190 used in laparoscopic surgery. FIG. 20 is an image of a commercially available single site multi-port system for laparoscopic surgery. FIG. 21 is an image of a commercially available articulating laparoscope 210.

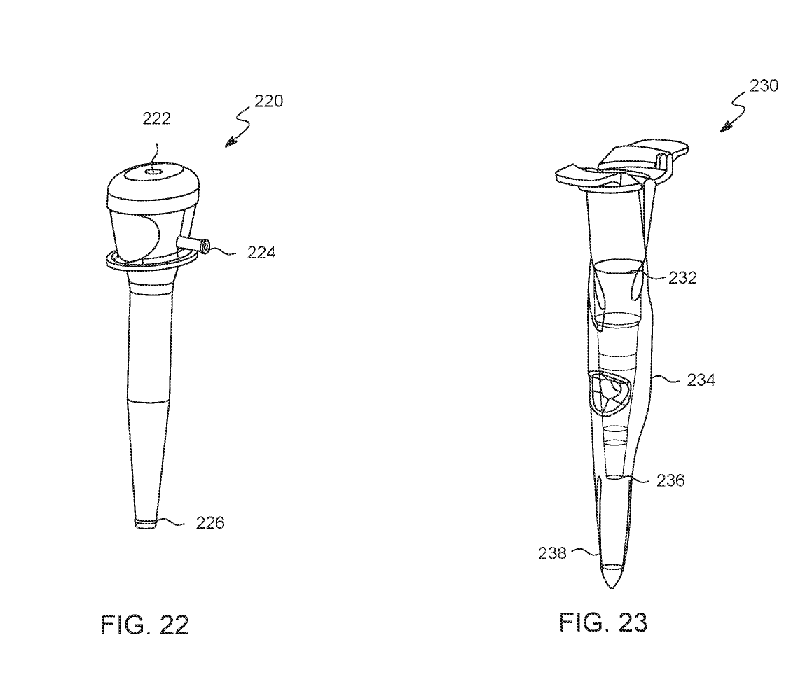

FIG. 22 illustrates a scope guide 220 that has a similar shape to the closure guide described above, however it could have a central lumen 222 (also referred to as the scope lumen 222) down the center that allows for passage of a scope 210, along with a fitting 224, such as a Luer that communicates with an inflation lumen, which could be the same lumen as the scope lumen 222. The scope lumen 222 could have a seal 228 (i.e., a duckbill seal) located above the access hole from the fitting 224 to prevent gas flow from exiting the proximal (top) end of the scope lumen 222. At the distal end, the scope guide 220 could have a seal 226 that seals against a surface of the adaptor 230 (see FIG. 23) when the scope guide 220 is securely positioned in the adaptor 230. The obturator/adaptor 230 could have a gas port 236 near the distal end of the inner cavity. It could also be constructed of a translucent or transparent material for optically viewing through the distal end. It could also have one or multiple scope rails 234 to facilitate guiding a laparoscope along the lateral side wall. This feature could also be a lumen to provide the same functionality. The obturator/adaptor 230 could also have a protruding lip 232 on the outer surface located just proximal to the relief 233 where the suture passing needle exits when placing suture. This protruding lip 232 could act as a tactile indicator that would be important for use when the abdominal wall in thin (i.e. in the umbilicus area). When the protruding lip 232 comes in contact with the skin, this would indicate the device was located at a depth where the needle will exit from the guide just below the skin. This same protruding lip feature could be on the closure guide and used in a similar fashion when the adaptor 230 is not used. The tip 238 of the obturator/adaptor 230 would have a geometric shape to facilitate dilating penetration through tissue for access through the tissue and into the abdominal cavity. The geometry could be fins, blades, tapered surface, cone, or other geometric shape to effectively dilate the tissue. The closure guide could have similar such geometric shape to facilitate dilating penetration through tissue for access through the tissue and into the abdominal cavity.

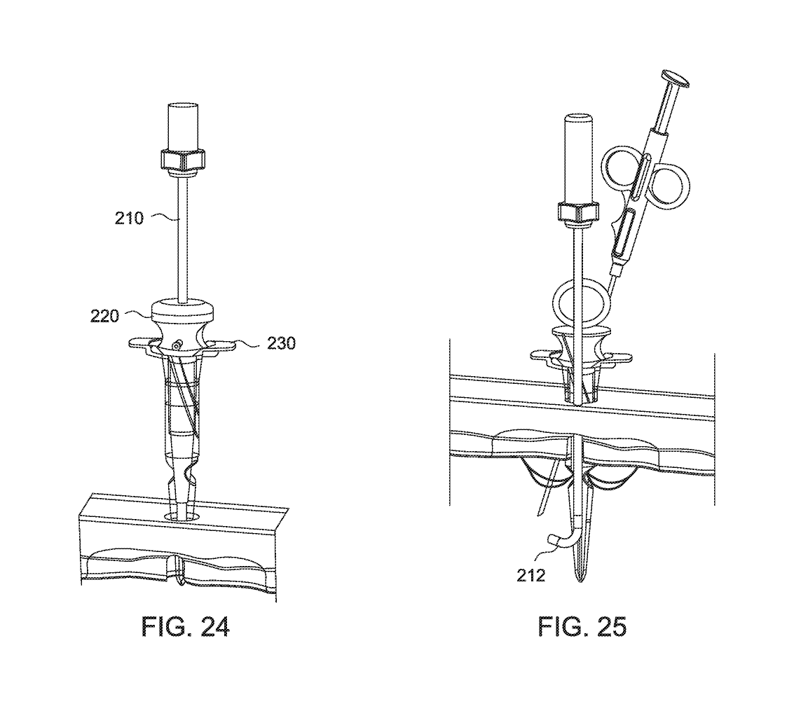

FIG. 24 illustrates the scope guide 220 securely mounted in the obturator/adaptor 230 and being penetrated through the tissue for access to the abdominal cavity. A scope 210 is positioned extending through the entire scope guide 220 and viewing into the distal end of the obturator/adaptor 230, being used in a similar manner to a conventional optical trocar. The fitting 224 (see FIG. 22) could be connected to a gas insufflation source, and when the obturator/adaptor 230 is positioned into the abdominal cavity where the gas port exit is beyond the peritoneum, thus enabling insufflation of the abdominal cavity. After insufflation is complete, the scope guide 220 could be removed while the obturator/adaptor 230 remains in the tissue. A closure guide 250 could then be securely mounted to the obturator/adaptor 230. An articulating scope 210, in the straight state, could be placed along the lateral side of the obturator/adaptor 230, utilizing the rail 234 to provide passage through the tissue. Once the articulating scope 210 is deep enough into the abdominal cavity, the distal end 212 of the scope 210 could be articulated in a direction to obtain adequate visualization for usage of the closure guide 250 and suture passer 252 to place and retrieve suture in the manner described above, for example, and as shown in FIG. 25. The retrieved suture tails could be tagged outside the body and left untied while the wound retractor, single site multiport system, or standard trocar is positioned in the site. The rest of the procedure is conducted and after the wound retractor, single site multiport system, or standard trocar is removed, the sutures could be tied to close the site. This technique is commonly known as a “pre-close” technique. It is understood that other techniques could be employed utilizing any combination of the four main components described above.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Claim elements and steps herein may have been numbered and/or lettered solely as an aid in readability and understanding. Any such numbering and lettering in itself is not intended to and should not be taken to indicate the ordering of elements and/or steps in the claims.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. An optical obturator/adaptor for laparoscopic procedures, comprising:
a tip configured to pass through tissue;
a hollow interior receiving one of a scope guide and a closure guide therein; and
a scope rail to facilitate guiding a laparoscope along the obturator/adaptor.

2. The optical obturator/adaptor of claim 1, wherein the scope rail is positioned along an outer edge of a lateral side wall thereof.

3. The optical obturator/adaptor of claim 1, wherein the scope rail is a lumen through the obturator/adaptor.

4. The optical obturator/adaptor of claim 1, wherein the tip is a bladeless tip.

5. The optical obturator/adaptor of claim 1, further comprising a protrusion disposed on an outer surface of the obturator/adaptor located proximal to a relief where a suture passing needle exits when placing a suture.

6. The optical obturator/adaptor of claim 1, further comprising a gas port near a distal end of the hollow interior.

7. The optical obturator/adaptor of claim 1, wherein the obturator/adaptor is formed from a translucent or transparent material for optically viewing through a distal end thereof.

8. A system for laparoscopic surgery, comprising:
an obturator/adaptor comprising:
a tip configured to pass through tissue;
a hollow interior; and
a scope rail to facilitate guiding a laparoscope along the obturator/adaptor; and
a scope guide receivable at least partially into the hollow interior of the obturator/adaptor, the scope guide comprising:
a central lumen for passage of a scope; and
a fitting communicating with an inflation lumen.

9. The system of claim 8, wherein the central lumen and the inflation lumen are a single lumen.

10. The system of claim 9, wherein the scope guide further comprises an inflation seal preventing gas flow from exiting a top end of the central lumen when the scope is inserted therethrough.

11. The system of claim 8, wherein the scope guide further comprises a lower seal pressing against a surface of the obturator/adaptor when the scope guide is securely positioned in the obturator/adaptor.

12. The system of claim 8, wherein the scope rail is positioned along an outer edge of a lateral side wall thereof.

13. The system of claim 8, further comprising a protrusion disposed on an outer surface of the obturator/adaptor located proximal to a relief where a suture passing needle exits when placing a suture.

14. The system of claim 8, further comprising a gas port near a distal end of the hollow interior, the gas port communicating with the inflation lumen of the scope guide.

15. A method for performing laparoscopic surgery, comprising:
inserting an obturator/adaptor through tissue for access to an abdominal cavity, the obturator/adaptor comprising a tip configured to pass through tissue, a hollow interior, and a scope rail to facilitate guiding a laparoscope;
mounting a scope guide into the obturator/adaptor, the scope guide comprising a central lumen for passage of a scope, and a fitting communicating with an inflation lumen;
connecting the fitting a gas insufflation source and, when a distal end of the obturator/adaptor is positioned into the abdominal cavity where a gas port exit is beyond the peritoneum, insufflating the abdominal cavity;
removing the scope guide while the obturator/adaptor remains in the tissue;
mounting a closure guide in the obturator/adaptor;
releasing snare loops from the closure guide;
placing a suture into each of the snare loops with a suture passer;
retracting the snare loops with suture ends captured therein against the closure guide; and
removing the closure guide to release the suture ends outside of the obturator/adaptor.

16. The method of claim 15, further comprising passing an articulating scope along the scope rail for visualizing the placement of the suture into each of the snare loops.

17. The method of claim 15, further comprising keeping the suture ends untied while performing the laparoscopic surgery.

18. The method of claim 17, further comprising tying ends of the suture to close the site.
